(19) United States
(12) Patent Application Publication
(10) Pub. No.: US 2014/0065682 A1
(43) Pub. Date: Mar. 6, 2014

(54) PROCESSES AND APPARATUS FOR PRODUCING FERMENTABLE SUGARS, CELLULOSE SOLIDS, AND LIGNIN FROM LIGNOCELLULOSIC BIOMASS

(71) Applicant: API Intellectual Property Holdings, LLC, Atlanta, GA (US)

(72) Inventors: Theodora RETSINA, Atlanta, GA (US); Vesa PYLKKANEN, Atlanta, GA (US); Kimberly NELSON, Atlanta, GA (US); Mark SZCZEPANIK, Alpena, MI (US); James Christian SAMP, Sugar Hill, GA (US)

(73) Assignee: API Intellectual Property Holdings, LLC, Atlanta, GA (US)

(21) Appl. No.: 14/017,286

(22) Filed: Sep. 3, 2013

Related U.S. Application Data
(60) Provisional application No. 61/696,360, filed on Sep. 4, 2012.

(51) Int. Cl. C13K 1/02; C12P 7/16; C12P 7/10

(52) U.S. Cl. CPC C13K 1/02; C12P 7/10; C12P 7/16
USPC 435/160; 127/37; 435/165

(57) ABSTRACT

Variations of this invention reduce or avoid lignin precipitation during acidic hydrolysis of biomass hydrolysates (such as hemicellulose-containing liquid extracts). Net acid usage and byproduct salt formation are significantly reduced. In some embodiments, hemicellulosic oligomers are hydrolyzed, in the presence of sulfur dioxide, to produce fermentable hemicellulosic sugars; the process comprising recovering and recycling at least a portion of the sulfur dioxide, wherein at least a portion of the sulfur dioxide reacts with the lignin to produce hydrophilic sulfonated lignin that has less tendency to precipitate or stick. In other embodiments, hemicellulosic oligomers are hydrolyzed, in the presence of a catalyst selected from the group consisting of sulfuric acid, sulfurous acid, sulfur dioxide, and combinations thereof, and an additive selected from metal sulfites, metal bisulfites, and combinations thereof, to produce fermentable hemicellulosic sugars, wherein at least a portion of the additive reacts with the lignin to produce sulfonated lignin.

### PRIORITY DATA

This patent application is a non-provisional application claiming priority to U.S. Provisional Patent App. No. 61/696,360, filed Sep. 4, 2012, which is hereby incorporated by reference herein.

### FIELD OF THE INVENTION

The present invention generally relates to improved processes for recovering fermentable sugars from lignocellulosic biomass.

### BACKGROUND OF THE INVENTION

Biomass refining (or biorefining), which separates cellulose, hemicellulose, and lignin from biomass feedstocks, is becoming more prevalent in industrial plants. Cellulose fibers and sugars, and hemicellulose sugars, are being used by many companies for chemical and fuel production. Indeed, we now are observing the commercialization of integrated biorefineries that are capable of processing incoming biomass much the same as petroleum refineries now process crude oil. Underutilized lignocellulosic biomass feedstocks have the potential to be much cheaper than petroleum, on a carbon basis, as well as much better from an environmental life-cycle standpoint.

One of the biggest and well-known challenges in many biorefineries is dealing with lignin. Lignin is a major component of biomass. It is typically between 15-35 wt % (dry basis) of the biomass material. Lignin has good fuel value, similar to some types of coal.

The word lignin is derived from the Latin word “lignum” meaning wood. Lignin is a natural polymer and is an essential part of wood and other forms of cellulosic biomass, including agricultural crop residues such as sugarcane bagasse. Lignin performs multiple functions that are essential to the life of the plant, including transport of nutrition and durability of the biomass. Lignin imparts rigidity to the cell walls and acts as a binder, creating a flexible composite cellulose-hemicellulose-lignin material that is outstandingly resistant to impact, compression, and bending.

After polysaccharides (polymers of sugar), lignin is the most abundant organic polymer in the plant world. Lignin is a very complex natural polymer with many random couplings, and therefore lignin has no exact chemical structure. The molecular structure of lignin consists primarily of carbon ring structures (benzene rings with methoxyl, hydroxyl, and propyl groups.

Various processes can be used to remove and isolate lignin from biomass. Each process, however, produces material of different composition and properties. Generally there are four important factors to take into account when working with lignin:
1. Source of the lignin.
2. Method used to remove lignin from the biomass.
3. Method(s) used to purify the lignin.
4. Nature of the chemical modification of the lignin after isolation.
   These factors influence the properties of the lignin. Important properties of lignin formulations include molecular weight, chemical composition, and the type and distribution of chemical functional groups.

Separation and recovery of lignin is quite difficult. It is possible to break the lignin-cellulose-hemicellulose matrix and recover the lignin through a variety of treatments on the lignocellulosic material. However, known lignin recovery methods generally have one or more important commercial-scale limitations. Lignin purification from biomass is a classic chemical-engineering problem with complex chemistries and transport phenomena, criticality of reactor design and scale-up, serious analytical challenges, and many practical issues arising from lignin's propensity to stick to equipment and piping.

Lignin can be difficult to process in biorefineries because it has a tendency to deposit on solid surfaces and cause plugging. Although lignin handling has always been known to be a challenge, there remains a need in the art for ways to either avoid lignin precipitation or to deal with it after it occurs. Other difficulties are caused by downstream fermentation inhibition caused by lignin, as well as lignin fragments and derivatives (e.g., phenolics, acids, and other compounds).

Lignin separations challenges appear to be particularly troubling problem for acidic pretreatments of biomass or biomass-derived liquors. For example, in van Heiningen et al., “Which fractionation process can overcome the techno-economic hurdles of a lignocellulosic biorefinery,” Proceedings of the AIChE Annual Meeting, Minneapolis, Minn. (2011), it is cautioned that “an operating problem which has mostly been overlooked for acidic pretreatment is formation and precipitation of sticky lignin on reactor walls and piping.” The lack of R&D attention to this problem is stated to be that it only “becomes apparent in continuous larger scale operation after one to two week operation.”

Another problem relating to acidic treatment of biomass is that after acid hydrolysis, the solution typically must be neutralized with a base, generating large quantities of a salt (such as gypsum). There is a need in the art to either reduce the amount of acid needed, or to be able to recover (remove) much of it prior to neutralization so that less salt byproduct is produced.

In view of the aforementioned needs in the art, improvements are needed to reduce, avoid, or deal with lignin precipitation during acidic hydrolysis of biomass and/or biomass hydrolysates (such as hemicellulose-containing liquid extracts). Improvements are also desired to reduce net acid usage or reduce byproduct salt formation. It would be preferred if improvements could address both lignin precipitation as well as salt formation.

### SUMMARY OF THE INVENTION

The present invention addresses the aforementioned needs in the art.

In some variations, the invention provides a process for producing fermentable hemicellulose sugars from lignocellulosic biomass, the process comprising:

(a) providing a feedstock comprising lignocellulosic biomass;

(b) extracting the feedstock with steam and/or hot water under effective extraction conditions to produce an extract liquor containing hemicellulosic oligomers, cellulose-rich solids, and lignin;

(c) substantially removing the cellulose-rich solids from the extract liquor;

(d) hydrolyzing the hemicellulosic oligomers contained in the extract liquor, in the presence of sulfur dioxide, to produce fermentable hemicellulosic sugars;

(e) recovering and recycling at least a portion of the sulfur dioxide from step (d); and

(f) recovering the fermentable hemicellulosic sugars from the extract liquor, wherein at least a portion of the sulfur dioxide reacts with the lignin to produce sulfonated lignin.

In some embodiments, the sulfonated lignin is hydrophilic and has reduced tendency to agglomerate, compared to the lignin. In some embodiments, the presence of the sulfonated lignin reduces precipitation of the lignin in the extract liquor.

In some embodiments, in step (d), the sulfur dioxide is present in a concentration of about 0.1 wt % to about 10 wt % of the extract liquor, such as about 0.5 wt % to about 2.5 wt % of the extract liquor. A portion of the sulfur dioxide may be present as sulfurous acid in the extract liquor. In certain embodiments, sulfur dioxide is generated in situ by introducing sulfurous acid, sulfite ions, bisulfite ions, combinations thereof, or a salt of any of the foregoing.

In step (d), the pH of the extract liquor may be adjusted to a pH from about 0 to about 2, for example. In some embodiments, the pH is adjusted by varying the concentration of the sulfur dioxide in the extract liquor. In these or other embodiments, the pH is adjusted by introducing a compound other than sulfur dioxide.

During or after step (f), the fermentable hemicellulosic sugars may be recovered in purified form, as a sugar slurry or dry sugar solids, for example.

In some embodiments, the process further comprises recovering the lignin as a co-product. The sulfonated lignin may also be recovered as a co-product. In certain embodiments, the process further comprises combusting or gasifying the sulfonated lignin, recovering sulfur contained in the sulfonated lignin in a gas stream comprising reclaimed sulfur dioxide, and then recycling the reclaimed sulfur dioxide back to step (d).

In some embodiments, the process further comprises removing a vapor stream comprising water and vaporized acetic acid from the extract liquor in at least one evaporation stage at a pH of 4.8 or less, to produce a concentrated extract liquor comprising the fermentable hemicellulosic sugars. At least one evaporation stage is preferably operated at a pH of 3.0 or less.

The process may further comprise a step of fermenting the fermentable hemicellulosic sugars to a fermentation product. The fermentation product may be ethanol, 1-butanol, isobutanol, or any other product (fuel or chemical).

In some embodiments, step (c) includes washing the cellulose-rich solids using an aqueous wash solution, to produce a wash filtrate; and optionally combining at least some of the wash filtrate with the extract liquor. Step (c) may further include pressing the cellulose-rich solids to produce the dewatered cellulose-rich solids and a press filtrate; and optionally combining at least some of the press filtrate with the extract liquor.

The disclosed process may further comprise combusting the cellulose-rich solids to produce power and/or heat. Alternatively, or additionally, the process may further comprise pelletizing the cellulose-rich solids to pellets for combustion, co-combustion with a fossil fuel, or gasification. Alternatively, or additionally, the process may include converting the cellulose-rich solids to a purified cellulose pulp, such as dissolving pulp.

In some variations, the invention provides a process for producing fermentable hemicellulose sugars from lignocellulosic biomass, the process comprising:

(a) providing a feedstock comprising lignocellulosic biomass;

(b) extracting the feedstock with steam and/or hot water, with a first amount of sulfur dioxide, under effective extraction conditions to produce an extract liquor containing hemicellulosic oligomers, cellulose-rich solids, and lignin;

(c) substantially removing the cellulose-rich solids from the extract liquor;

(d) hydrolyzing the hemicellulosic oligomers contained in the extract liquor, in the presence of a second amount of sulfur dioxide, to produce fermentable hemicellulosic sugars;

(e) recovering and recycling at least a portion of the sulfur dioxide from step (d); and

(f) recovering the fermentable hemicellulosic sugars from the extract liquor,

wherein at least a portion of the second amount of sulfur dioxide reacts with the lignin to produce sulfonated lignin.

The first amount of sulfur dioxide may include at least a portion of the second amount of sulfur dioxide that did not react with the lignin in step (d). In some embodiments, the second amount of sulfur dioxide is higher than the first amount of sulfur dioxide. In some embodiments, the sulfur dioxide concentration in step (d) is higher than the sulfur dioxide concentration in step (b).

The sulfonated lignin is hydrophilic and has reduced tendency to agglomerate, compared to the starting lignin, in preferred embodiments. The presence of the sulfonated lignin may reduce precipitation of the lignin in the extract liquor.

In some embodiments, in step (b), the sulfur dioxide is present in a concentration of about 0.01 wt % to about 3 wt % of the extract liquor. In certain embodiments, in step (b), the sulfur dioxide is present in a concentration of about 0.1 wt % to about 1 wt % of the extract liquor. In some embodiments, in step (d), the sulfur dioxide is present in a concentration of about 0.1 wt % to about 10 wt % of the extract liquor. In certain embodiments, in step (d), the sulfur dioxide is present in a concentration of about 0.5 wt % to about 2.5 wt % of the extract liquor.

In step (d), the pH of the extract liquor may be adjusted to a pH from about 0 to about 2, for example. pH adjustment may be accomplished by varying the concentration of the sulfur dioxide in the extract liquor and/or by introducing a compound (e.g., acid, base, or buffer) other than sulfur dioxide. A portion of the sulfur dioxide may be present as sulfurous acid in the extract liquor. In some embodiments, the sulfur dioxide is generated in situ by introducing sulfurous acid, sulfite ions, bisulfite ions, combinations thereof, or a salt of any of the foregoing.

In some other variations of the invention, a process for producing fermentable hemicellulose sugars from lignocellulosic biomass comprises the steps of:

(a) providing a feedstock comprising lignocellulosic biomass;

(b) extracting the feedstock with steam and/or hot water under effective extraction conditions to produce an extract liquor containing hemicellulosic oligomers, cellulose-rich solids, and lignin;

(c) substantially removing the cellulose-rich solids from the extract liquor;

(d) hydrolyzing the hemicellulosic oligomers contained in the extract liquor, in the presence of (i) a catalyst selected from the group consisting of sulfuric acid, sulfurous acid, sulfur dioxide, and combinations thereof, and (ii) an additive selected from metal sulfites, metal bisulfites, and combinations thereof, to produce fermentable hemicellulosic sugars; and

(e) recovering the fermentable hemicellulosic sugars,

wherein at least a portion of the additive reacts, directly or indirectly, with the lignin to produce sulfonated lignin.

The presence of the additive reduces precipitation of the lignin in the extract liquor, in preferred embodiments. The sulfonated lignin is hydrophilic and may have reduced tendency to agglomerate, compared to the starting lignin.

In some embodiments, in step (d), the catalyst is present in a concentration of about 0.1 wt % to about 10 wt % of the extract liquor. In certain embodiments, in step (d), the catalyst is present in a concentration of about 0.5 wt % to about 3 wt % of the extract liquor.

In some embodiments, in step (d), the additive is present in a concentration of about 100 ppm to about 10,000 ppm of the extract liquor. In certain embodiments, in step (d), the additive is present in a concentration of about 200 ppm to about 5,000 ppm of the extract liquor.

The pH of the extract liquor may be adjusted from about 0 to about 2 in some embodiments. Adjustment of pH may be accomplished by varying the concentration of the catalyst and/or the additive in the extract liquor. In some embodiments, the pH is adjusted by introducing a compound other than the catalyst or the additive.

In some embodiments, the catalyst includes sulfur dioxide, or consists essentially of sulfur dioxide. In some embodiments, the additive includes sodium sulfite and/or sodium bisulfite. In some embodiments, the additive includes potassium sulfite and/or potassium bisulfite. The additive may be generated in situ by introducing a base to react a portion of the catalyst with the base to form the additive, if desired. The process of some embodiments includes recovering and recycling at least a portion of the catalyst(s) and/or additive(s).

In some embodiments, during or after step (f), the fermentable hemicellulosic sugars are recovered in purified form as a sugar slurry or dry sugar solids. In some embodiments, lignin and/or sulfonated lignin is recovered as co-product(s).

The process may include removing a vapor stream comprising water and vaporized acetic acid from the extract liquor in at least one evaporation stage at a pH of 4.8 or less, to produce a concentrated extract liquor comprising the fermentable hemicellulosic sugars. At least one evaporation stage may be operated at a pH of 3.0 or less.

The process of this variation may further comprise a step of fermenting the fermentable hemicellulosic sugars to a fermentation product, such as (but not limited to) ethanol, 1-butanol, isobutanol, or combinations thereof.

Step (c) may include washing the cellulose-rich solids using an aqueous wash solution, to produce a wash filtrate; and optionally combining at least some of the wash filtrate with the extract liquor. In some embodiments, step (c) further includes pressing the cellulose-rich solids to produce the dewatered cellulose-rich solids and a press filtrate; and optionally combining at least some of the press filtrate with the extract liquor.

The process may include combusting the cellulose-rich solids to produce power and/or heat; pelletizing the cellulose-rich solids to pellets for combustion, co-combustion with a fossil fuel, or gasification; and/or converting the cellulose-rich solids to a purified cellulose pulp.

The invention, in some variations, provides a process for producing fermentable hemicellulose sugars from lignocellulosic biomass, the process comprising:

(a) providing a feedstock comprising lignocellulosic biomass;

(b) extracting the feedstock with steam and/or hot water under effective extraction conditions to produce an extract liquor containing hemicellulosic oligomers, cellulose-rich solids, and lignin;

(c) substantially removing the cellulose-rich solids from the extract liquor;

(d) hydrolyzing the hemicellulosic oligomers contained in the extract liquor, in the presence of a catalyst mixture of (i) SO₂ and/or H₂SO₃, and (ii) SO₃²⁻, in sulfite anion or salt form and/or HSO₃⁻, in bisulfite anion or salt form, to produce fermentable hemicellulosic sugars; and

(e) recovering the fermentable hemicellulosic sugars.

The presence of the additive reduces precipitation of the lignin in the extract liquor, in preferred embodiments. When the catalyst mixture includes metal sulfites, the metal sulfites may be selected from sodium sulfite or potassium sulfite. When the catalyst mixture includes metal bisulfites, the metal bisulfites may be selected from sodium bisulfite or potassium bisulfite.

The catalyst mixture may be adjusted to control the pH of the extract liquor to a pH of from about 0 to about 2, without limitation. In some embodiments, the composition and/or pH of the catalyst mixture is adjusted to control the concentration of free SO₂ dissolved in the extract liquor. In some embodiments, the composition and/or pH of the catalyst mixture is adjusted to control the concentration of SO₃²⁻, in anion form. In some embodiments, the composition and/or pH of the catalyst mixture is adjusted to control the concentration of HSO₃⁻, in anion form. Preferably, the process is controlled to minimize release of SO₂ vapors.

Other variations provide a process for producing fermentable hemicellulose sugars from lignocellulosic biomass, the process comprising:

(a) providing a feedstock comprising lignocellulosic biomass;

(b) extracting the feedstock with steam and/or hot water, with sulfur dioxide, under effective extraction conditions to produce an extract liquor containing hemicellulosic oligomers, cellulose-rich solids, and lignin;

(c) substantially removing the cellulose-rich solids from the extract liquor;

(d) hydrolyzing the hemicellulosic oligomers contained in the extract liquor, in the presence of an additive selected from metal sulfites, metal bisulfites, and anions or combinations thereof, to produce fermentable hemicellulosic sugars; and

(e) recovering the fermentable hemicellulosic sugars.

Preferably, the presence of the additive reduces precipitation of the lignin in the extract liquor. In some embodiments, at least a portion of the sulfur dioxide from step (b) is passed to step (d) for hydrolyzing the hemicellulosic oligomers.

The present invention also provides systems configured for carrying out the disclosed processes, and compositions produced therefrom.

### BRIEF DESCRIPTION OF THE FIGURES

FIG. 1 is a simplified block-flow diagram depicting the process of some embodiments of the present invention.

FIG. 2 is a simplified block-flow diagram depicting the process of some embodiments of the present invention.

### DETAILED DESCRIPTION OF EMBODIMENTS OF THE INVENTION

This description will enable one skilled in the art to make and use the invention, and it describes several embodiments, adaptations, variations, alternatives, and uses of the invention. These and other embodiments, features, and advantages of the present invention will become more apparent to those skilled in the art when taken with reference to the following detailed description of the invention in conjunction with any accompanying drawings.

As used in this specification and the appended claims, the singular forms “a,” “an,” and “the” include plural referents unless the context clearly indicates otherwise. Unless defined otherwise, all technical and scientific terms used herein have the same meaning as is commonly understood by one of ordinary skill in the art to which this invention belongs. All composition numbers and ranges based on percentages are weight percentages, unless indicated otherwise. All ranges of numbers or conditions are meant to encompass any specific value contained within the range, rounded to any suitable decimal point.

Unless otherwise indicated, all numbers expressing reaction conditions, stoichiometries, concentrations of components, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phase “consisting essentially of” limits the scope of a claim to the specified elements or method steps, plus those that do not materially affect the basis and novel characteristic(s) of the claimed subject matter.

With respect to the terms “comprising,” “consisting of,” and “consisting essentially of,” where one of these three terms is used herein, the presently disclosed and claimed subject matter may include the use of either of the other two terms. Thus in some embodiments not otherwise explicitly recited, any instance of “comprising” may be replaced by “consisting of” or, alternatively, by “consisting essentially of.”

Some variations of the invention are premised on the realization that sulfur dioxide may be a preferred sulfur-containing acid catalyst, or precursor thereof, for hydrolyzing biomass hemicellulosic extracts. There are several potential reasons, without being limited to any particular theory or hypothesis.

First, it is believed that sulfur dioxide is a more-efficient catalyst for catalyzing hydrolysis reactions to convert hemicellulose oligomers to monomers. Sulfur dioxide at ambient conditions is a gas which will have higher mass-transfer rates within a hydrolysis reactor, leading to more uniform hydrolysis chemistry. It is thought that in order for SO₂ to function as a hydrolysis catalyst, it must proceed through a reactive intermediate that contains a proton (H⁺). After the reaction step, the proton may be returned to solution and molecular SO₂ regenerated.

In particular, SO₂ in water will normally convert to some extent to sulfurous acid, H₂SO₃ (which exists in solution as H⁺ and HSO₃) whose dissociated hydrogen atom may initiate the reaction. The reaction hydrolysis starts with a proton from sulfurous acid interacting rapidly with a glycosidic oxygen linking two sugar units, forming a conjugate acid. The cleavage of the C—O bond and breakdown of the conjugate acid to the cyclic carbonium ion then takes place. After a rapid addition of a molecule of water, free sugar and a proton are liberated. That proton must return to the starting acid, H₂SO₃, or to the water phase. Stoichiometrically, another way to view these reactions is that SO₂ temporarily combines with water, which is added to the sugar polymers to hydrolyze them (necessarily consuming a water molecule). In turn, the SO₂ is again available for further chemistry, or recovery from the reactor prior to the reactor contents moving downstream. Recovery is made easier since the SO₂ molecule is very volatile.

This increased efficiency owing to the inherent properties of sulfur dioxide mean that less acid may be required. This has cost advantages itself, since sulfuric acid can be expensive. Additionally, and quite significantly, less acid usage also will translate into lower costs for a base (e.g., lime) to increase the pH following hydrolysis, for downstream operations. Furthermore, less acid and less base will also mean substantially less generation of waste salts (e.g., gypsum) that may otherwise require disposal.

Another reason that sulfur dioxide may be preferred relates not to sugar hydrolysis chemistry, but to lignin chemistry. It has been surprisingly discovered, through lab-scale experiments, that acid hydrolysis of hemicellulose with sulfur dioxide leads to dramatically less lignin deposition, compared to acid hydrolysis with sulfuric acid, for the same final sugar yield.

Without being limited by any theory, it is believed that SO₂ (or HSO₃⁻) can react directly with lignin to produce sulfonated lignin (also known as lignosulfonates). The reaction of sulfur dioxide or a bisulfite ion with lignin is thought to involve acidic cleavage of ether bonds, which connect many of the constituents of lignin. The electrophilic carbocations produced during ether cleavage react with bisulfite ions to give lignosulfonates. An important site for ether cleavage is the α-carbon (carbon atom attached to the aromatic ring) of the propyl side chain of lignin. Sulfur dioxide does not tend to catalyze condensation reactions of lignin that increase molecular weight. Mechanistically, acid-catalyzed condensation and sulfonation can involve the same carbon atom, the α-carbon of the propyl group. The implication is that SO₂ or HSO₃ may directly react with this carbon atom before condensation reactions can be initiated.

Also, native (non-sulfonated) lignin is hydrophobic, while lignosulfonates are hydrophilic. Hydrophilic lignosulfonates may have less propensity to clump, agglomerate, and stick to surfaces. Even lignosulfonates that do undergo some condensation and increase of molecular weight, will still have an HSO₃ group that will contribute some solubility (hydrophilic).

Another reason that sulfur dioxide may be a preferred acid catalyst, or precursor thereof, is that SO₂ can be recovered easily from solution after hydrolysis. The majority of the SO₂ from the hydrolysate may be stripped and recycled back to the reactor. Recovery and recycling translates to less lime required compared to neutralization of comparable sulfuric acid, less solids to dispose of, and less separation equipment.

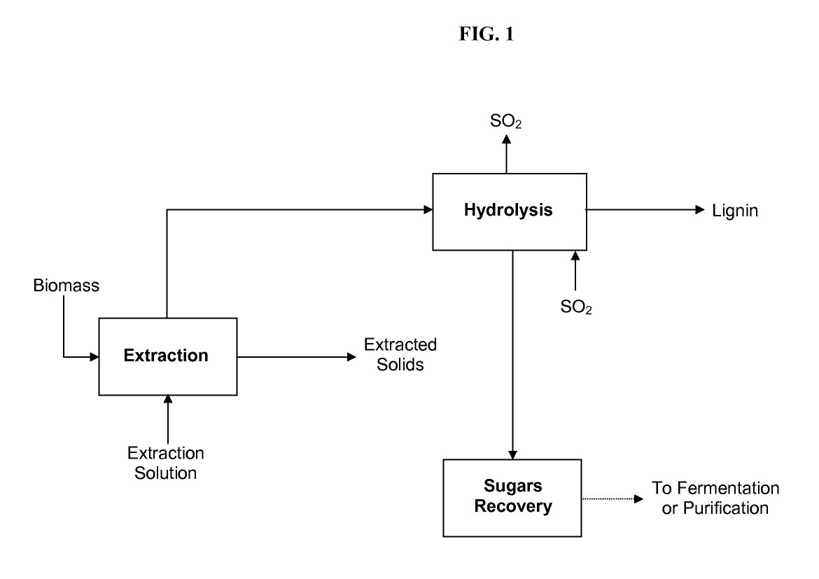

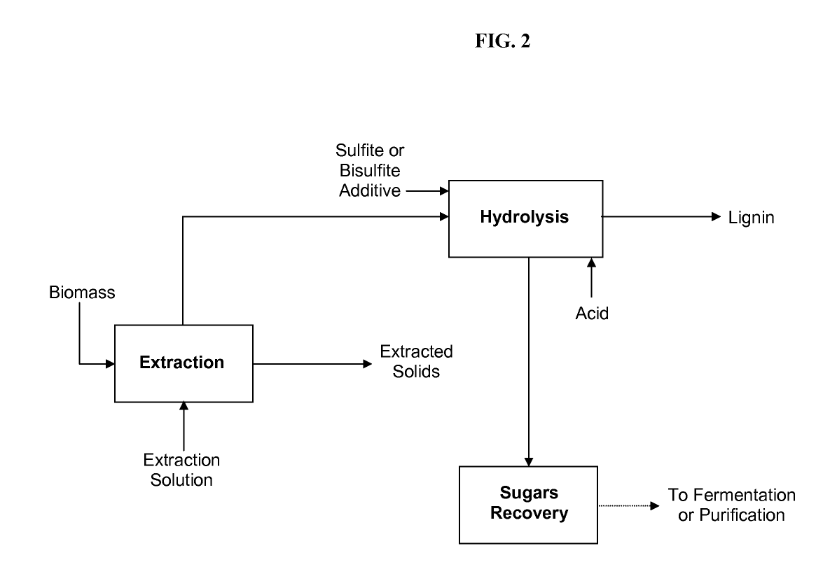

Certain exemplary embodiments of the invention will now be described. These embodiments are not intended to limit the scope of the invention as claimed. The order of steps may be varied, some steps may be omitted, and/or other steps may be added. Reference herein to first step, second step, etc. is for illustration purposes only. Some embodiments can be understood with reference to FIGS. 1 and 2. Dotted lines in are optional streams.

In some variations relating to FIG. 1, the invention provides a process for producing fermentable hemicellulose sugars from lignocellulosic biomass, the process comprising:

(a) providing a feedstock comprising lignocellulosic biomass;

(b) extracting the feedstock with steam and/or hot water under effective extraction conditions to produce an extract liquor containing hemicellulosic oligomers, cellulose-rich solids, and lignin;

(c) substantially removing the cellulose-rich solids from the extract liquor;

(d) hydrolyzing the hemicellulosic oligomers contained in the extract liquor, in the presence of sulfur dioxide, to produce fermentable hemicellulosic sugars;

(e) recovering and recycling at least a portion of the sulfur dioxide from step (d); and

(f) recovering the fermentable hemicellulosic sugars from the extract liquor,

wherein at least a portion of the sulfur dioxide reacts with the lignin to produce sulfonated lignin.

The biomass feedstock may be selected from hardwoods, softwoods, forest residues, industrial wastes, consumer wastes, or combinations thereof. Some embodiments utilize agricultural residues, which include lignocellulosic biomass associated with food crops, annual grasses, energy crops, or other annually renewable feedstocks. Exemplary agricultural residues include, but are not limited to, corn stover, corn fiber, wheat straw, sugarcane bagasse, rice straw, oat straw, barley straw, miscanthus, energy cane, or combinations thereof.

In some embodiments, the sulfonated lignin is hydrophilic and has reduced tendency to agglomerate, compared to the lignin. In some embodiments, the presence of the sulfonated lignin reduces precipitation of the lignin in the extract liquor.

Reaction conditions and operation sequences in steps (a)-(d) may vary widely. Some embodiments employ conditions described in U.S. patent application Ser. Nos. 13/471,662; 13/026,273; 13/026,280; 13/500,917; 61/536,477; 61/612,451; 61/612,453; 61/624,880; 61/638,730; and 61/641,435. Each of these commonly owned patent applications are hereby incorporated by reference herein in their entireties.

Effective extraction conditions may include contacting the lignocellulosic biomass with steam (at various pressures in saturated, superheated, or supersaturated form) and/or hot water. In some embodiments, the process is a variation of the Green Power+™ process technology which is commonly owned with the assignee of this patent application.

In some embodiments, in step (d), the sulfur dioxide is present in a concentration of about 0.1 wt % to about 10 wt % of the extract liquor, such as about 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, or 2.5 wt % of the extract liquor. A portion or all of the sulfur dioxide may be present as sulfurous acid in the extract liquor. In certain embodiments, sulfur dioxide is generated in situ by introducing sulfurous acid, sulfite ions, bisulfite ions, combinations thereof, or a salt of any of the foregoing. Excess sulfur dioxide, following hydrolysis, may be recovered and reused.

In some embodiments, sulfur dioxide is saturated in water (or aqueous solution) at a first temperature, and the hydrolysis is then carried out at a second, generally higher, temperature. In some embodiments, sulfur dioxide is sub-saturated. In some embodiments, sulfur dioxide is super-saturated. In some embodiments, sulfur dioxide concentration is selected to achieve a certain degree of lignin sulfonation, such as 1%, 2%, 3%, 4%, 5%, 6%, 7%, 8%, 9%, or 10% sulfur content.

In step (d), the pH of the extract liquor may be adjusted to a pH from about −2 to 4, such as to about −1.0, −0.5, 0.0, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 3.5, or 4.0, for example. In some embodiments, the pH is adjusted by varying the concentration of the sulfur dioxide in the extract liquor. In these or other embodiments, the pH is adjusted by introducing a compound other than sulfur dioxide.

In step (e), recovering and recycling the sulfur dioxide may utilize separations such as, but not limited to, vapor-liquid disengagement (e.g. flashing), steam stripping, extraction, or combinations or multiple stages thereof. Various recycle ratios may be practiced, such as about 0.1, 0.2, 0.3, 0.4, 0.5, 0.6, 0.7, 0.8, 0.9, 0.95, or more (calculated as ratio of recycled SO₂ to total SO₂ charged to hydrolysis reactor).

During or after step (f), the fermentable hemicellulosic sugars may be recovered in purified form, as a sugar slurry or dry sugar solids, for example. Any known technique may be employed to recover a slurry of sugars or to dry the solution to produce dry sugar solids.

In some embodiments, the process further comprises recovering the lignin as a co-product. The sulfonated lignin may also be recovered as a co-product. In certain embodiments, the process further comprises combusting or gasifying the sulfonated lignin, recovering sulfur contained in the sulfonated lignin in a gas stream comprising reclaimed sulfur dioxide, and then recycling the reclaimed sulfur dioxide back to step (d).

In some embodiments, the process further comprises removing a vapor stream comprising water and vaporized acetic acid from the extract liquor in at least one evaporation stage at a pH of 4.8 or less, to produce a concentrated extract liquor comprising the fermentable hemicellulosic sugars. At least one evaporation stage is preferably operated at a pH of 3.0 or less.

The process may further comprise a step of fermenting the fermentable hemicellulosic sugars to a fermentation product. The fermentation product may be ethanol, 1-butanol, isobutanol, or any other product (fuel or chemical). Some amount of the fermentation product may be growth of a microorganism or enzymes, which may be recovered if desired.

In some embodiments, the fermentable hemicellulose sugars are recovered from solution, in purified form. In some embodiments, the fermentable hemicellulose sugars are fermented to produce of biochemicals or biofuels such as (but by no means limited to) ethanol, 1-butanol, isobutanol, acetic acid, lactic acid, or any other fermentation products. A purified fermentation product may be produced by distilling the fermentation product, which will also generate a distillation bottoms stream containing residual solids. A bottoms evaporation stage may be used, to produce residual solids.

In some embodiments, step (c) includes washing the cellulose-rich solids using an aqueous wash solution, to produce a wash filtrate; and optionally combining at least some of the wash filtrate with the extract liquor. Step (c) may further include pressing the cellulose-rich solids to produce the dewatered cellulose-rich solids and a press filtrate; and optionally combining at least some of the press filtrate with the extract liquor.

The disclosed process may further comprise combusting the cellulose-rich solids to produce power and/or heat. Alternatively, or additionally, the process may further comprise pelletizing the cellulose-rich solids to pellets for combustion, co-combustion with a fossil fuel, or gasification. Alternatively, or additionally, the process may include converting the cellulose-rich solids to a purified cellulose pulp, such as dissolving pulp.

In some variations, the invention provides a process for producing fermentable hemicellulose sugars from lignocellulosic biomass, the process comprising:

(a) providing a feedstock comprising lignocellulosic biomass;

(b) extracting the feedstock with steam and/or hot water, with a first amount of sulfur dioxide, under effective extraction conditions to produce an extract liquor containing hemicellulosic oligomers, cellulose-rich solids, and lignin;

(c) substantially removing the cellulose-rich solids from the extract liquor;

(d) hydrolyzing the hemicellulosic oligomers contained in the extract liquor, in the presence of a second amount of sulfur dioxide, to produce fermentable hemicellulosic sugars;

(e) recovering and recycling at least a portion of the sulfur dioxide from step (d); and

(f) recovering the fermentable hemicellulosic sugars from the extract liquor,

wherein at least a portion of the second amount of sulfur dioxide reacts with the lignin to produce sulfonated lignin.

The first amount of sulfur dioxide may include at least a portion of the second amount of sulfur dioxide that did not react with the lignin in step (d). In some embodiments, the second amount of sulfur dioxide is higher than the first amount of sulfur dioxide. In some embodiments, the sulfur dioxide concentration in step (d) is higher than the sulfur dioxide concentration in step (b).

In step (e), recovering and recycling at least a portion of the second amount of sulfur dioxide may utilize separations such as, but not limited to, vapor-liquid disengagement (e.g. flashing), steam stripping, extraction, or combinations or multiple stages thereof. Various recycle ratios may be practiced, such as about 0.1, 0.2, 0.3, 0.4, 0.5, 0.6, 0.7, 0.8, 0.9, 0.95, or more (calculated as ratio of recycled SO₂ to total SO₂ charged to hydrolysis reactor).

The sulfonated lignin is hydrophilic and has reduced tendency to agglomerate, compared to the starting lignin, in preferred embodiments. The presence of the sulfonated lignin may reduce precipitation of the lignin in the extract liquor.

In some embodiments, in step (b), the sulfur dioxide is present in a concentration of about 0.01 wt % to about 3 wt % of the extract liquor, such as about 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, or 2.5 wt %. In certain embodiments, in step (b), the sulfur dioxide is present in a concentration of about 0.1 wt % to about 1 wt % of the extract liquor.

In some embodiments, in step (d), the sulfur dioxide is present in a concentration of about 0.1 wt % to about 10 wt % of the extract liquor, such as about 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 3.0, 4.0, 5.0, 6.0, 7.0, 8.0, or 9.0 wt %. In certain embodiments, in step (d), the sulfur dioxide is present in a concentration of about 0.5 wt % to about 2.5 wt % of the extract liquor.

In step (d), the pH of the extract liquor may be adjusted to a pH from about 0 to about 2, such as to about −1.0, −0.5, 0.0, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 3.5, or 4.0, for example. pH adjustment may be accomplished by varying the concentration of the sulfur dioxide in the extract liquor and/or by introducing a compound (e.g., acid, base, or buffer) other than sulfur dioxide. A portion of the sulfur dioxide may be present as sulfurous acid in the extract liquor. In some embodiments, the sulfur dioxide is generated in situ by introducing sulfurous acid, sulfite ions, bisulfate ions, combinations thereof, or a salt of any of the foregoing.

Other variations of the invention (such as shown in FIG. 2) are premised on the use of metal sulfites and/or metal bisulfites as additives, in addition to an acid catalyst (which may or may not be SO₂). The sulfite/bisulfite additives can produce lignosulfonates and prevent lignin from extensive condensation, in a similar fashion as described earlier. Sulfonic groups attached to the lignin may increase the hydrophilicity of the residual lignin. Also, it is believed that in some embodiments sulfite/bisulfite additives may effectively depolymerize lignin, to some extent, thereby reversing acid-catalyzed condensation that may have taken place.

In some other variations of the invention, a process for producing fermentable hemicellulose sugars from lignocellulosic biomass comprises the steps of:

(a) providing a feedstock comprising lignocellulosic biomass;

(b) extracting the feedstock with steam and/or hot water under effective extraction conditions to produce an extract liquor containing hemicellulosic oligomers, cellulose-rich solids, and lignin;

(c) substantially removing the cellulose-rich solids from the extract liquor;

(d) hydrolyzing the hemicellulosic oligomers contained in the extract liquor, in the presence of (i) a catalyst selected from the group consisting of sulfuric acid, sulfurous acid, sulfur dioxide, and combinations thereof, and (ii) an additive selected from metal sulfites, metal bisulfites, and combinations thereof, to produce fermentable hemicellulosic sugars; and

(e) recovering the fermentable hemicellulosic sugars,

wherein at least a portion of the additive reacts, directly or indirectly, with the lignin to produce sulfonated lignin.

The biomass feedstock may be selected from hardwoods, softwoods, forest residues, industrial wastes, consumer wastes, or combinations thereof. Some embodiments utilize agricultural residues, which include lignocellulosic biomass associated with food crops, annual grasses, energy crops, or other annually renewable feedstocks. Exemplary agricultural residues include, but are not limited to, corn stover, corn fiber, wheat straw, sugarcane bagasse, rice straw, oat straw, barley straw, miscanthus, energy cane, or combinations thereof.

The presence of the additive reduces precipitation of the lignin in the extract liquor, in preferred embodiments. The sulfonated lignin is hydrophilic and may have reduced tendency to agglomerate, compared to the starting lignin.

Reaction conditions and operation sequences in steps (a)-(d) may vary widely. Some embodiments employ conditions described in U.S. patent application Ser. Nos. 13/471,662; 13/026,273; 13/026,280; 13/500,917; 61/536,477; 61/612,451; 61/612,453; 61/624,880; 61/638,730; and 61/641,435. Each of these commonly owned patent applications are hereby incorporated by reference herein in their entireties.

Effective extraction conditions may include contacting the lignocellulosic biomass with steam (at various pressures in saturated, superheated, or supersaturated form) and/or hot water. In some embodiments, the process is a variation of the Green Power+™ process technology which is commonly owned with the assignee of this patent application.

In some embodiments, in step (d), the catalyst is present in a concentration of about 0.1 wt % to about 10 wt % of the extract liquor, such as about 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, or 2.5 wt %. In certain embodiments, in step (d), the catalyst is present in a concentration of about 0.5 wt % to about 3 wt % of the extract liquor.

In some embodiments, in step (d), the additive is present in a concentration of about 100 ppm to about 10,000 ppm of the extract liquor, such as about 200, 300, 400, 500, 750, 1,000, 2,000, 3,000, 4,000, 5,000, 6,000, 7,000, 8,000, 9,000 ppm. In certain embodiments, in step (d), the additive is present in a concentration of about 200 ppm to about 5,000 ppm of the extract liquor. Less than 100 ppm or more than 10,000 ppm (1 wt %) additive may be employed, in some embodiments.

The pH of the extract liquor may be adjusted from about 0 to about 2 in some embodiments, such as to about −1.0, −0.5, 0.0, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 3.5, or 4.0, for example. Adjustment of pH may be accomplished by varying the concentration of the catalyst and/or the additive in the extract liquor. In some embodiments, the pH is adjusted by introducing a compound other than the catalyst or the additive. When high additive concentrations are utilized, the acid concentration may need to be increased to overcome pH buffering effects.

In some embodiments, the catalyst includes sulfur dioxide, or consists essentially of sulfur dioxide. In some embodiments, the additive includes sodium sulfite and/or sodium bisulfite. In some embodiments, the additive includes potassium sulfite and/or potassium bisulfite. The additive may be generated in situ by introducing a base to react a portion of the catalyst with the base to form the additive, if desired. The process of some embodiments includes recovering and recycling at least a portion of the catalyst(s) and/or additive(s).

In some embodiments, during or after step (f), the fermentable hemicellulosic sugars are recovered in purified form as a sugar slurry or dry sugar solids. In some embodiments, lignin and/or sulfonated lignin is recovered as co-product(s).

The process may include removing a vapor stream comprising water and vaporized acetic acid from the extract liquor in at least one evaporation stage at a pH of 4.8 or less, to produce a concentrated extract liquor comprising the fermentable hemicellulosic sugars. At least one evaporation stage may be operated at a pH of 3.0 or less.

The process of this variation may further comprise a step of fermenting the fermentable hemicellulosic sugars to a fermentation product, such as (but not limited to) ethanol, 1-butanol, isobutanol, or combinations thereof.

Step (c) may include washing the cellulose-rich solids using an aqueous wash solution, to produce a wash filtrate; and optionally combining at least some of the wash filtrate with the extract liquor. In some embodiments, step (c) further includes pressing the cellulose-rich solids to produce the dewatered cellulose-rich solids and a press filtrate; and optionally combining at least some of the press filtrate with the extract liquor.

The process may further comprise recovering and recycling at least a portion of the sulfur dioxide, at least a portion of the additive, or both.

The process may include combusting the cellulose-rich solids to produce power and/or heat; pelletizing the cellulose-rich solids to pellets for combustion, co-combustion with a fossil fuel, or gasification; and/or converting the cellulose-rich solids to a purified cellulose pulp.

The invention, in some variations, provides a process for producing fermentable hemicellulose sugars from lignocellulosic biomass, the process comprising:

(a) providing a feedstock comprising lignocellulosic biomass;

(b) extracting the feedstock with steam and/or hot water under effective extraction conditions to produce an extract liquor containing hemicellulosic oligomers, cellulose-rich solids, and lignin;

(c) substantially removing the cellulose-rich solids from the extract liquor;

(d) hydrolyzing the hemicellulosic oligomers contained in the extract liquor, in the presence of a catalyst mixture of (i) SO₂ and/or H₂SO₃, and (ii) SO₃²⁻, in sulfite anion or salt form and/or HSO₃⁻, in bisulfite anion or salt form, to produce fermentable hemicellulosic sugars; and

(e) recovering the fermentable hemicellulosic sugars.

The presence of the additive reduces precipitation of the lignin in the extract liquor, in preferred embodiments. When the catalyst mixture includes metal sulfites, the metal sulfites may be selected from sodium sulfite or potassium sulfite. When the catalyst mixture includes metal bisulfites, the metal bisulfites may be selected from sodium bisulfite or potassium bisulfite.

The catalyst mixture may be adjusted to control the pH of the extract liquor to a pH of from about 0 to about 2, without limitation. In some embodiments, the composition and/or pH of the catalyst mixture is adjusted to control the concentration of free SO₂ dissolved in the extract liquor. In some embodiments, the composition and/or pH of the catalyst mixture is adjusted to control the concentration of SO₃²⁻, in anion form. In some embodiments, the composition and/or pH of the catalyst mixture is adjusted to control the concentration of HSO₃⁻, in anion form. Preferably, the process is controlled to minimize release of SO₂ vapors.

Other variations provide a process for producing fermentable hemicellulose sugars from lignocellulosic biomass, the process comprising:

(a) providing a feedstock comprising lignocellulosic biomass;

(b) extracting the feedstock with steam and/or hot water, with sulfur dioxide, under effective extraction conditions to produce an extract liquor containing hemicellulosic oligomers, cellulose-rich solids, and lignin;

(c) substantially removing the cellulose-rich solids from the extract liquor;

(d) hydrolyzing the hemicellulosic oligomers contained in the extract liquor, in the presence of an additive selected from metal sulfites, metal bisulfites, and anions or combinations thereof, to produce fermentable hemicellulosic sugars; and

(e) recovering the fermentable hemicellulosic sugars.

Preferably, the presence of the additive reduces precipitation of the lignin in the extract liquor. In some embodiments, at least a portion of the sulfur dioxide from step (b) is passed to step (d) for hydrolyzing the hemicellulosic oligomers.

The present invention also provides systems configured for carrying out the disclosed processes, and compositions produced therefrom. Any stream generated by the disclosed processes may be partially or completed recovered, purified or further treated, and/or marketed or sold.

In this detailed description, reference has been made to multiple embodiments of the invention and non-limiting examples relating to how the invention can be understood and practiced. Other embodiments that do not provide all of the features and advantages set forth herein may be utilized, without departing from the spirit and scope of the present invention. This invention incorporates routine experimentation and optimization of the methods and systems described herein. Such modifications and variations are considered to be within the scope of the invention defined by the claims.

All publications, patents, and patent applications cited in this specification are herein incorporated by reference in their entirety as if each publication, patent, or patent application were specifically and individually put forth herein.

Where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially.

Therefore, to the extent there are variations of the invention, which are within the spirit of the disclosure or equivalent to the inventions found in the appended claims, it is the intent that this patent will cover those variations as well. The present invention shall only be limited by what is claimed.

### EXAMPLE 1

A laboratory study was performed to determine if hydrolysis of extract liquor at 4% solids can be accomplished using SO₂ rather than sulfuric acid.

Procedure for SO₂ hydrolysis at 270° F. for 1 hour

1. Acquire packing and set up pilot digester as absorption column.

2. Design experiment that will demonstrate effectiveness of sulfur dioxide absorption of liquor.
a. Precool 10 liter digester to 0° C. by filling with water and ice.
b. Prepare 10 liters of 4% liquor cooled to 32° F. and saturated to 3% sulfur dioxide.
c. Fill digester bleeding air/SO₂ to safe location.
d. Close up reactor, begin circulation, begin digester heating.
e. At 40° C. drain and dispose of 6 liters from the reactor circuit creating an SO₂ gas phase in the top of the digester. Continue heating.
f. Digester pressure and temperature recorded at 5 minute intervals during warmup and during hydrolysis.
g. Digester pressure controlled to maximum of 200 psig as necessary by venting SO₂ to safe location. Record all vent times.
h. Pull small samples for determination of hydrolyzate sugar and acids content in HPLC at following intervals.
   1.) Liquor circulating prior to heating
   2.) Liquor circulating once at temperature
   3.) Every 5 minutes for first 20 minutes once digester at target temperature
   4.) Every 10 minutes thereafter for next 40 minutes ending at one hour
i. Complete duplicate analysis of all samples to determine hydrolyzate sugar and acids content in HPLC.

First hydrolysis test used a Parr bomb reactor (2 L reactor with 1 L working liquid, 1 L void volume) with 3 wt % SO₂ charge, pH ˜0. The 4% solids liquor charged with SO₂ at 0° C., and then hydrolyzed at 132° C. for 1 hour, shaken periodically. The heating time is 30 minutes to temperature from 0° C., the pressure increased to 150 psig, then back down, held at 70 psig.

A second hydrolysis test used a Parr bomb reactor (2 L reactor with 1 L working liquid, 1 L void volume) with saturated SO₂ charge (10 minutes), pH ˜0.4. The 4% solids liquor charged with SO₂ at 80° C. Liquor then hydrolyzed at 145° C. for 1 hour, shaken periodically. The heating time is 18 minutes to temperature from 80° C., the pressure increased to 100 psig, then back down, held at 70 psig. Precipitation is very light on the reactor surface.

Both conditions produced sugars while leaving very little lignin residue adhered to the Parr digester walls. These tests indicate that hydrolysis using SO₂ has benefits of lower reactor deposition. The use of SO₂ also enables recovery and reuse of unreacted sulfur dioxide at the conclusion of the hydrolysis.

Table 1 compares the sugars produced from a sulfuric acid hydrolysis method and from the two SO₂ saturation methods.

TABLE 1

| | SO₂ | Sugar Concen- | |
|---|---|---|---|
| | Solubility, | tration After | |
| | g SO₂/ | Hydrolysis, | Hydrolysis |
| | L water | mg/ml | Conditions |
| H₂SO₄ at 302° F. | NA | 22.6 | 302° F., 60 minutes, |
| | | | and 1.0 wt % sulfuric |
| | | | acid charge |
| H₂SO₄ at 248° F. | NA | 16.0 | 248° F., 15 minutes, |
| (APB design basis) | | | and 1.0 wt % sulfuric |
| | | | acid charge |
| SO₂ with saturation | 232 | 16.2 | 270° F., 60 minutes, |
| at 270° F. | | | 3% SO₂ charge, pH |
| | | | of 0.0 |
| SO₂ with saturation | 21.3 | 13.8 | 293° F., 60 minutes, |
| at 293° F. | | | unknown SO₂ |
| | | | charge, pH of 0.4 |

Significantly lower disposition is observed with SO₂ hydrolysis. The loose and non-sticky precipitate from SO₂ hydrolysis is easily removed with lime.

### EXAMPLE 2

Liquor at 4.2 wt % solids is combined with 200 ppm and 5,000 ppm sodium sulfite preheated to 250° F. in a Parr reactor. At 250° F., 1% sulfuric acid is injected to liquor and hydrolysis is performed for 1 hour. Reactor then cooled slowly in air until 206° F. and opened.

5,000 ppm Sodium Sulfite

Hydrolysis with 5,000 ppm sodium sulfite was very successful at giving a clean reactor with no fouling on walls or on the temperature probe, similar to Example 1. Lignin was light orange color rather than black, indicating uncondensed/minimally condensed form.

200 ppm Sodium Sulfite

Hydrolysis with 200 ppm sodium sulfite gave a thin, black, glassy precipitate on the reactor walls and temperature probe. Lignin was a mixture of orange and black colors, indicating presence of more condensed lignin. Lignin poured from the bottom of reactor behaved like a mixture of gritty material in water. With 200 ppm sodium sulfite, fouling was reduced compared to hydrolysis with no additive, but lignin fouling was not eliminated.

Sodium Sulfate Concentration

| | 200 ppm | 5000 ppm |
|---|---|---|
| pH | 1.19 | 1.54 |
| Glucose | 1.308 | 0.346 |
| Xylose | 7.461 | 5.337 |
| Galactose | 1.663 | 1.126 |
| Arabinose | 4.169 | 4.039 |
| Mannose | 0.867 | 0.482 |
| Total | 15.468 | 11.33 g/L |

What is claimed is:

1. A process for producing fermentable hemicellulose sugars from lignocellulosic biomass, said process comprising:
(a) providing a feedstock comprising lignocellulosic biomass;
(b) extracting said feedstock with steam and/or hot water under effective extraction conditions to produce an extract liquor containing hemicellulosic oligomers, cellulose-rich solids, and lignin;
(c) substantially removing said cellulose-rich solids from said extract liquor;
(d) hydrolyzing said hemicellulosic oligomers contained in said extract liquor, in the presence of sulfur dioxide, to produce fermentable hemicellulosic sugars;
(e) recovering and recycling at least a portion of said sulfur dioxide from step (d); and
(f) recovering said fermentable hemicellulosic sugars from said extract liquor,
wherein at least a portion of said sulfur dioxide reacts with said lignin to produce sulfonated lignin.

2. The process of claim 1, wherein said sulfonated lignin is hydrophilic and has reduced tendency to agglomerate, compared to said lignin.

3. The process of claim 1, wherein the presence of said sulfonated lignin reduces precipitation of said lignin in said extract liquor.

4. The process of claim 1, wherein in step (d), said sulfur dioxide is present in a concentration of about 0.1 wt % to about 10 wt % of said extract liquor.

5. The process of claim 1, wherein said sulfur dioxide is generated in situ by introducing sulfurous acid, sulfite ions, bisulfate ions, combinations thereof, or a salt of any of the foregoing.

6. The process of claim 1, wherein during or after step (f), said fermentable hemicellulosic sugars are recovered in purified form as a sugar slurry or dry sugar solids.

7. The process of claim 1, said process further comprising recovering said lignin as a co-product.

8. The process of claim 1, said process further comprising recovering said sulfonated lignin as a co-product.

9. The process of claim 8, said process further comprising combusting or gasifying said sulfonated lignin, recovering sulfur contained in said sulfonated lignin in a gas stream comprising reclaimed sulfur dioxide, and then recycling said reclaimed sulfur dioxide back to step (d).

10. The process of claim 1, said process further comprising a step of fermenting said fermentable hemicellulosic sugars to a fermentation product.

11. The process of claim 1, said process further comprising combusting said cellulose-rich solids to produce power and/or heat.

12. The process of claim 1, said process further comprising pelletizing said cellulose-rich solids to pellets for combustion, co-combustion with a fossil fuel, or gasification.

13. The process of claim 1, said process further comprising converting said cellulose-rich solids to a purified cellulose pulp.

14. A process for producing fermentable hemicellulose sugars from lignocellulosic biomass, said process comprising:
(a) providing a feedstock comprising lignocellulosic biomass;
(b) extracting said feedstock with steam and/or hot water under effective extraction conditions to produce an extract liquor containing hemicellulosic oligomers, cellulose-rich solids, and lignin;
(c) substantially removing said cellulose-rich solids from said extract liquor;
(d) hydrolyzing said hemicellulosic oligomers contained in said extract liquor, in the presence of (i) a catalyst selected from the group consisting of sulfuric acid, sulfurous acid, sulfur dioxide, and combinations thereof, and (ii) an additive selected from metal sulfites, metal bisulfites, and combinations thereof, to produce fermentable hemicellulosic sugars; and
(e) recovering said fermentable hemicellulosic sugars,
wherein at least a portion of said additive reacts, directly or indirectly, with said lignin to produce sulfonated lignin.

15. The process of claim 14, wherein the presence of said additive reduces precipitation of said lignin in said extract liquor.

16. The process of claim 14, wherein said sulfonated lignin is hydrophilic and has reduced tendency to agglomerate, compared to said lignin.

17. The process of claim 14, wherein in step (d), said catalyst is present in a concentration of about 0.1 wt % to about 10 wt % of said extract liquor.

18. The process of claim 14, wherein in step (d), said additive is present in a concentration of about 100 ppm to about 10,000 ppm of said extract liquor.

19. The process of claim 14, wherein said additive is generated in situ by introducing a base to react a portion of said catalyst with said base to form said additive.

20. The process of claim 14, said process further comprising recovering and recycling at least a portion of said catalyst.

21. The process of claim 14, said process further comprising recovering and recycling at least a portion of said additive.

22. The process of claim 14, wherein during or after step (f), said fermentable hemicellulosic sugars are recovered in purified form as a sugar slurry or dry sugar solids.

23. The process of claim 14, said process further comprising recovering said lignin as a co-product.

24. The process of claim 14, said process further comprising recovering said sulfonated lignin as a co-product.

25. The process of claim 14, said process further comprising a step of fermenting said fermentable hemicellulosic sugars to a fermentation product.

26. The process of claim 14, said process further comprising combusting said cellulose-rich solids to produce power and/or heat.

27. The process of claim 14, said process further comprising pelletizing said cellulose-rich solids to pellets for combustion, co-combustion with a fossil fuel, or gasification.

28. The process of claim 14, said process further comprising converting said cellulose-rich solids to a purified cellulose pulp.
